(19) United States
(12) Patent Application Publication
(10) Pub. No.: US 2010/0224200 A1
(43) Pub. Date: Sep. 9, 2010

(54) Infant Restraining Article

(76) Inventor: Megan Kopczynski, South Bend, IN (US)

Correspondence Address: L.C. Begin & Associates, PLLC, 510 Highland Avenue, PMB 403, Milford, MI, 48381, US

(21) Appl. No.: 12/715,433

(22) Filed: Mar. 2, 2010

Related U.S. Application Data
(60) Provisional application No. 61/157,326, filed on Mar. 4, 2009.

(51) Int. Cl. A61F 5/37

(52) U.S. Cl. 128/870

(57) ABSTRACT

One embodiment of an infant restraining article includes a base member that includes a pad, and one or more safety straps attached to the pad. The one or more safety straps are capable of securing the pad to an article, such as a bedding article. Further, the infant restraining article includes a jacket member attached to the pad for configuring a resting space for an infant. The jacket member includes a pair of flap members adapted to receive arms of the infant and to removably engage to each other for enclosing the infant in the resting space. The enclosing of the infant in the resting space restricts free movement of the infant.

### CROSS REFERENCE TO RELATED APPLICATION

This application claims the benefit of U.S. Provisional Application No. 61/157,326 filed on Mar. 4, 2009 the disclosure of which is incorporated by reference.

### FIELD OF THE DISCLOSURE

The present disclosure generally relates to an infant restraining article, and, more particularly, to an article that restricts free movement of an infant during sleep.

### BACKGROUND OF THE DISCLOSURE

To avoid Sudden Infant Death Syndrome (SIDS) or crib death, doctors recommend that parents ensure their baby sleeps in a supine posture. However, following such a recommendation may be a tedious and disturbing exercise for the parents. Following such a recommendation may require the parents to constantly monitor the sleeping posture of their baby. This may additionally require the parents to wake up at odd hours in the night to supervise the posture. For convenience, parents may be inclined to bring their infant into their own bed to monitor its posture. This practice, known as “bed-sharing” has resulted in infant death, because of suffocation and/or overheating, due to the parents' own radiation of body heat, or, due to the parents inadvertently rolling over the infant.

Many attempts have been made to develop restraining articles that restrict free movement of babies, especially during sleep. Usage of such conventional restraining articles may ensure that the babies do not turn over onto their stomachs during sleep. Further, the use of such conventional restraining articles precludes the need of constantly monitoring the babies.

However, the conventional restraining articles have proven to be inadequate in some respects. The conventional restraining articles, when used, may be uncomfortable for the babies. Further, the conventional restraining articles have been unable to keep the babies warm. As a result, warmers, such as blankets, may still be required to be used in addition to the conventional restraining articles.

Furthermore, the conventional restraining articles usually restrain the babies in a way that makes it difficult for the parents to change their clothes or diapers. Moreover, the conventional restraining articles may not be adapted to fit the babies as the babies grow older. This may necessitate the purchase of larger and larger sized restraining articles, leading to unnecessary expenditure by the parents. Lastly, the conventional restraining articles may be bulky and difficult to carry.

### SUMMARY OF THE INVENTION

In accordance with one embodiment of the present invention an infant restraining article includes a base member that contains a pad, and one or more safety straps carried by one or more peripheral edge portions of the pad. The one or more safety straps are capable of securing the pad to a bedding article. Further, the infant restraining article includes a jacket member attached to a surface of the pad for configuring a resting space for an infant. The jacket member comprises a pair of flap members adapted to receive arms of the infant therein and also adapted to be removably engaged or secured with each other for enclosing the infant in the resting space. The enclosing of the infant in the resting space thereby restricts free movement of the infant.

### BRIEF DESCRIPTION OF THE DRAWINGS

These and other features and advantages of the present disclosure will be apparent from the following detailed description of preferred embodiments and best mode, appended claims, and accompanying drawings in which:

FIG. 1 is a side view of one embodiment of an infant restraining article;

FIG. 2 is a side view of the infant restraining article of FIG.1 attached to a bedding article;

FIG. 3 is a back view of the infant restraining article of FIG. 1; and

FIG. 4 is a top view of the infant restraining article of FIG. 1 in use.

### DETAILED DESCRIPTION

The detailed exemplary embodiments described herein are for illustrative purposes only, and are subject to many variations in structure and design. It should be emphasized, therefore, that the present disclosure is not limited to a particular infant restraining article, as shown and described. It is understood that various omissions and substitutions of equivalents are contemplated as circumstances may suggest or render expedient, but these are intended to cover the application or embodiment without departing from the spirit or scope of the claims of the present disclosure. Also, it is to be understood that the phraseology and terminology used herein are for the purpose of description and should not be regarded as limiting.

The use of “including,” “comprising,” or “having” and variations thereof herein is meant to encompass the items listed thereafter and equivalents thereof as well as additional items. The terms such as “first,” “second,” “outer,” “inner” and other variations thereof as used herein, do not denote an order, elevation or importance, but rather used signify the presence of at least one of the referenced item. Further, the terms, “a” and “an” herein do not denote a limitation of quantity, but rather denote the presence of at least one of the referenced item.

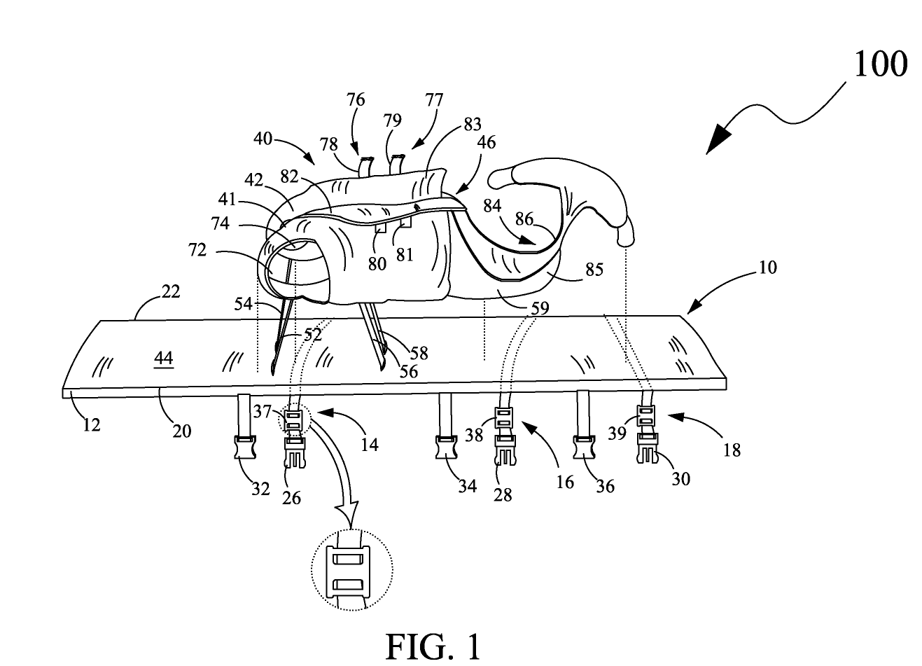

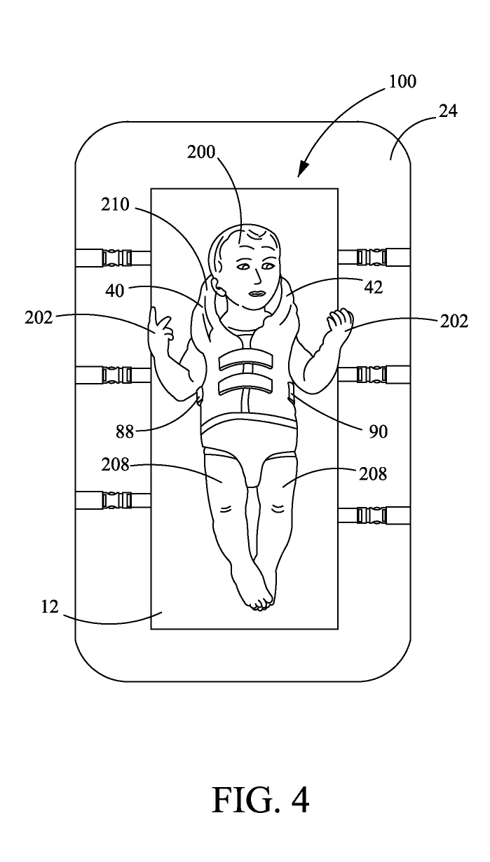

Referring to FIGS. 1-4 there is shown one embodiment of an infant restraining article 100 used for restricting free movement of an infant 200.

The infant restraining article 100 includes a base member 10. The base member 10 includes a pad 12 capable of cushioning the infant 200. The pad 12 may be similar to baby pads currently known in the art. Further, the pad 12 may be composed of an outer sheet having soft pre-sterilized materials stuffed in the outer sheet. However, the soft pre-sterilized materials may be stuffed into the outer sheet in such a manner so as to impart a degree of rigidity to pad 12. This may reduce chances of the pad 12 curling up or otherwise deforming when used. For this purpose, the pad 12 may include a thin plank of plywood, polymer, or some other suitable material. Moreover, the use of soft materials ensures that, when used, the pad 12 does not cause any irritation to the infant 200. Further, the use of sterilized materials helps ensure that the pad 12 is free of any germs and mites.

Further, the base member 10 may include one or more safety straps 14, 16, 18 carried by peripheral edge portions 20, 22 of the pad 12. More specifically, the safety straps 14, 16, 18 may be sewn or otherwise suitably attached to the peripheral edge portions 20, 22 of the pad 12. However, it should be understood that such sewing of the safety straps 14, 16, 18 to the pad 12 should not be construed as a limitation to the present disclosure. Alternatively, the safety straps 14, 16, 18 may be hooked to the peripheral edge portions 20, 22, or attached to the peripheral edge portions 20, 22 using other removable attachment means, such as clips or Velcro® fasteners. Such removable attachment may ensure that the safety straps 14, 16, 18 may be removed from the pad 12 by a user, if desired.

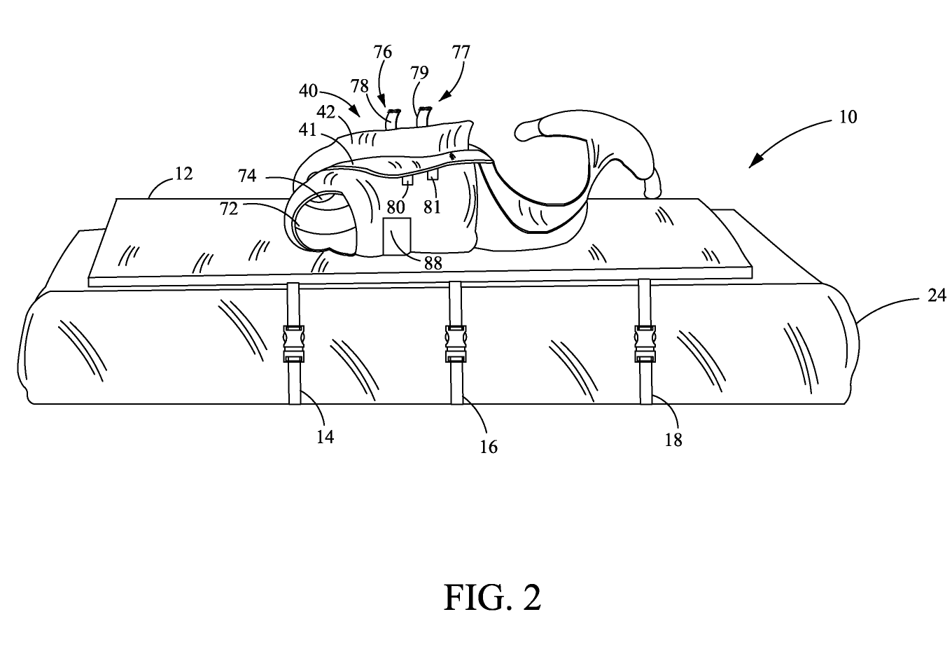

The safety straps 14, 16, 18 are capable of securing the pad 12 to a bedding article 24 (as shown in FIG. 2). The safety straps 14, 16, 18 may be similar to straps existing known in the art. More specifically, the safety straps 14, 16, 18 may include male buckling elements 26, 28, 30 and female buckling elements 32, 34, 36. When the pad 12 is required to be secured to the bedding article 24, the safety straps 14, 16, 18 may be wrapped around the bedding article 24 and the male buckling elements 26, 28, 30 may be removably engaged to the corresponding female buckling elements 32, 34, 36. More particularly, the male buckling elements 26, 28, 30 may be snap engaged/snap fit within the corresponding female buckling elements 32, 34, 36; and then side portions of the male buckling elements 26, 28, 30 may be pressed to disengage from the female buckling elements 32, 34, 36. Moreover, length of the safety straps 14, 16, 18 may be adjustable by providing loop clips 37, 38, 39 adjacent to the male buckling elements 26, 28, 30. Such adjustment of the safety straps 14, 16 and 18 may provide a freedom to the user in securing the pad 12, and thereby the infant restraining article 100, to almost any size bedding article 24.

It should be understood that the bedding article 24, in the embodiment, is shown to be an infant mattress. Alternatively, the bedding article 24 may be any other suitable bedding article, such as a cushion or other similar article.

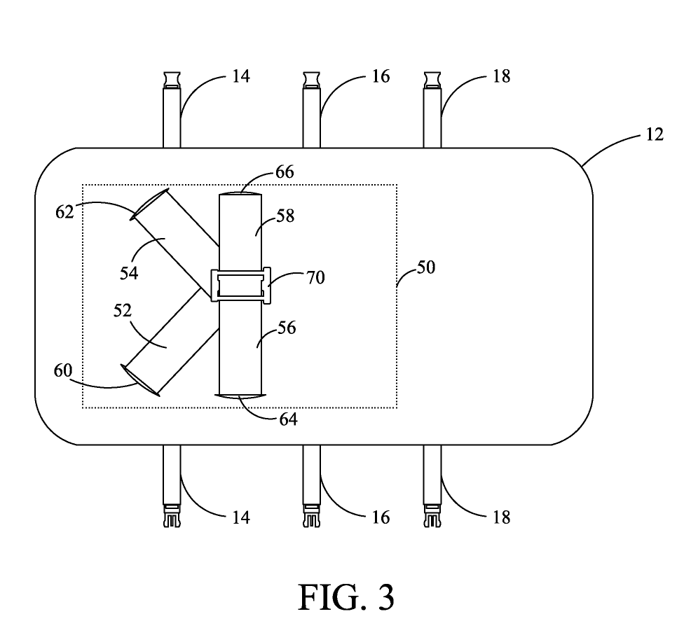

The infant restraining article 100 may further include a jacket member 40 attached to a surface of the pad 12 for configuring a resting space for the infant. The jacket member 40 may comprise a pair of flap members 41, 42 attached to a surface 44 of the pad 12. The attaching of the flap members 41, 42 to the surface 44 configures a resting space 46. In one embodiment, the resting space 46 may be adjusted according to the size of the infant 200. For this purpose, the flap members 41, 42 may be adjustably attached to the surface 44 of the pad 12 using an adjusting mechanism 50 (shown in the back view of the infant restraining article 100 in FIG. 3).

The adjusting mechanism 50 includes one or more fit straps 52, 54, 56, 58 extending from, or carried by, bottom portion 59 of the jacket member 40. The fit straps 52, 54, 56, 58 may be received in respective slots 60, 62, 64, 66 provided on the pad 12. The fit straps 52, 54 may be secured to one another in a cross pattern (as shown in FIG. 3), while the fit straps 56, 58 are looped into a loop clip 70. The securing of the fit straps 52, 54 with each other and the looping of the fit straps 56, 58 into the loop clip 70 allow an easy adjustment of the resting space 46 in accordance with the size of the infant 200. However, such adjustable attaching of the jacket member 40 to the pad 12 should not be construed as a limitation to the present disclosure. Alternatively, the jacket member 40 may be sewn to the surface 44 of the pad 12, or may be attached to the surface 44 of the pad 12 by any other means, such as Velcro® or other suitable fasteners, stitching, pins, clips, or any other suitable means.

The flap members 41, 42 may further include one or more openings 72, 74. The openings 72, 74 are configured to receive arms 202 of the infant 200 therethrough. Such receiving of the arms 202 in the openings 72, 74 helps to enclose the infant 200 in the resting space 46 of the infant restraining article 100 while permitting the arms of the infant to move freely

Further, the flap members 41, 42 may also include a pair of fastening members 76, 77 (shown to be in unfastened position). More specifically, the pair of fastening members 76, 77 may be carried by, and specifically attached, to slacking portions 82, 83 of the flap members 41, 42. Each of the fastening members 76, 77 comprises an engagement element 78, 79 and a complementary engaging element 80, 81. The engagement element 78, 79 is capable of being removably engaged with the complementary engagement element 80, 81 for removably engaging the pair of flap members 41, 42 with each other. This restricts free movement of the infant 200 only to the resting space 46. In the embodiment shown, the fastening members 76, 77 may be hook and loop fasteners, such as a Velcro® fasteners. More specifically, hook sides of the Velcro® (engagement element 78, 79) may be attached on the flap member 41, while the loop sides of the Velcro® (complementary engagement element 80, 81) may be attached to the flap member 42. Alternatively, other fasteners, such as clips, zippers, hooks, or other such fastening articles may also be used as the fastening members 76, 77.

Each of the flap members 41, 42 may extend outwards from the openings 72, 74 to configure a leg receiving section 84. The leg receiving portion 84 is capable of receiving legs 208 of the infant 200, and thereafter the leg receiving portion 84 may be folded inwardly to removably attach to the slacking portions 82, 83 of the flap members 41, 42. Such receiving of the legs 208 in the leg receiving section 84 further restricts the free movement of the infant 200. More specifically, the flap members 41, 42 may extend outwards from the openings 72, 74 into raised portions 85, 86, thereby configuring the leg receiving section 84 in the infant restraining article 100.

The jacket member 40 may be composed of very soft, warm materials. The use of soft materials prevents rough contact of the jacket member 40 with skin of the infant 200. Further, the use of warm materials precludes the need of using separate warmers in conjunction with the infant restraining article 100. In one embodiment, the jacket member 40 may be composed of materials which are a blend of polyester and cotton. Alternatively, the jacket member 40 may also be composed of pure cotton materials, polyester materials, soft wool materials, soft thermal insulation materials, and other similar or useful materials.

Further, the jacket member 40 may have different sizes. The jacket member 40 may have a length less than length of torso of the infant 200. This provides freedom to the parents to change diapers of the infant 200 without removing the infant 200 from the infant restraining article 100. Also, the flap members 41, 42, when attached to each other for enclosing the infant 200, may create a wide collar 210 around neck of the infant 200. Such a wide collar 210 provides clearance or spacing from the neck of the infant.

In addition to the base member 10 and the flap members 40, 42, the infant restraining article 100 may further include one or more underarm straps 88, 90. The underarm straps 88, 90 may be similar to a rectangular patch, and may be used to secure the flap members 40, 42 to the pad 12. Such securing may prevent or aid in preventing movement of the flap members 40, 42 off of the pad 12. Accordingly, the use of the underarm straps 88, 90 may ensure that the infant 200, when received in the infant restraining article 100, does not slouch. This may prevent any unnecessary movement of the infant 200 in the infant restraining article 100.

It should be understood that various components of the infant restraining article 100, as described above, may be manufactured in different customized sizes and shapes. For example, the pad 12 may be manufactured to be of circular shape rather than rectangular shape (as shown in FIGS. 1-4). Further, the size of the flap members 41, 42 may vary depending upon age and/or size of the infant 200. In addition, it should be understood that the various components of the infant restraining article 100 may also have different designs or other features imprinted on them. Therefore, such variations of the infant restraining article 100 should not be construed as outside the scope of the present disclosure.

In one embodiment, the infant restraining article 100 may be collapsible for making it portable. To elaborate, the jacket member 40 may be detached from the pad 12. Further, after detachment, the pad 12 may be folded about an axis. Such folding may provide portability to the infant restraining article 100. Alternatively, the flap members 41, 42 of the jacket member 40 may be adapted to be foldable over the pad 12 for folding the infant restraining article 100.

In use, the infant restraining article 100 may be first secured to the bedding article 24. More specifically, the pad 12 of the infant restraining article 100 may be secured to the bedding article 24 by wrapping the safety straps 14, 16, 18 around the bedding article 24, and by coupling the male buckling elements 26, 28, 30 to the female buckling elements 32, 34, 36. Thereafter, the resting space 46, configured between the flap members 41, 42 of the jacket member 40, may be adjusted according to the size of the infant 200. The adjustment may be done using the adjusting mechanism 50. More specifically, lengths of the straps 56, 58 received in the loop clip 70 may be adjusted to adjust the resting space 46.

After the resting space 46 is adjusted, the infant 200 may be placed in the resting space 46. More specifically, the arms 202 of the infant 200 may be received in the openings 72, 74. Thereafter, the fastening elements 76, 78 may be fastened, thereby wrapping the flap members 41, 42 on the infant 200, and more specifically, across the chest area of the infant 200. This encloses the infant 200 in the resting space 46, thereby restricting the free movement of the infant 200. Further, this precludes any possibility of the infant 200 turning on to his/her stomach, when restrained in the infant restraining article 100. After use, the infant restraining article 100 may be folded by folding the flap members 41, 42 on the pad 12, or by detaching the flap members 41, 42 from the pad 12.

The present disclosure provides an infant restraining article, such as infant restraining article 100. The infant restraining article includes abase member having a pad, and one or more safety straps carried by the pad. Further, the infant restraining article includes a pair of flap members attached to a surface of the pad. The attachment of the pair of flap members configures a resting space, where an infant can be received. Further, the pair of flap members may be adapted to receive the infant, and attach to each other for enclosing the infant. The infant restraining article is composed of soft, warm and sterilized materials. As a result, use of the infant restraining article is soft on skin of the infants, free from germs and may provide sufficient insulation to the infant secured in the infant restraining article. Further, the infant restraining article is simple in construction and may be collapsible, thereby making it portable. Also, the infant restraining article provides a more comfortable fit to the infants because of its wide-collared design. In addition, the infant restraining article may allow parents to easily change the clothes or diapers of the infant. Also, the size of the resting space is adjustable to adapt to the growing size of the infant. This leads to savings for the parents as they do not need to buy larger and larger-sized restraining articles.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present disclosure.

What is claimed is:

1. An infant restraining article, comprising:
a base member including a pad and at least one safety strap attached to the pad, the one or more safety straps being configured for securing the pad to an article; and
a jacket member attached to the pad and configured to define a resting space for an infant, the jacket member comprising a pair of flap members adapted to receive arms of the infant therethrough and to removably engage each other for enclosing the infant in the resting space.

2. The infant restraining article of claim 1, wherein the at least one safety strap comprises at least one pair of safety straps, one strap of the at least one pair of straps including a male buckling element and one strap of the at least one pair of straps including a female buckling element, and wherein the male buckling element is capable of being removably engaged to the female buckling element for securing the pad to the bedding article.

3. The infant restraining article of claim 1, wherein the pair of flap members comprises:
at least one pair of openings for receiving the arms of the infant; and
a pair of fastening members carried by the pair of flap members, each of the fastening members comprising one of an engagement element and a complementary engaging element, wherein the engagement element is capable of being removably engaged with the complementary engagement element for removably engaging the pair of flap members to each other.

4. The infant restraining article of claim 1, wherein the pair of flap members extends outwardly into a raised portion to configure a leg receiving section.

5. The infant restraining article of claim 1, further comprising a pair of underarm straps, each underarm strap of the pair of underarm straps being capable of securing a flap member of the pair of flap members to the surface of the pad, to prevent movement of the flap member of the pair of flap members off of the pad.

6. The infant restraining article of claim 1, further comprising an adjusting mechanism for adjustably attaching the pair of flap members to the surface of the pad.

7. The infant restraining article of claim 6, wherein the adjusting mechanism comprises one or more fit straps attached to the pair of flap members, the one or more fit straps adapted to adjustably attach the pair of flap members to the surface of the pad.

8. The infant restraining article of claim 1, wherein the jacket member comprises a polyester based material.

9. The infant restraining article of claim 1, wherein the jacket member comprises a blend of cotton-based material and polyester-based material.
